(19) United States
(12) Patent Application Publication
(10) Pub. No.: US 2021/0151185 A1
(43) Pub. Date: May 20, 2021

(54) INFORMATION PROCESSING DEVICE, INFORMATION PROCESSING SYSTEM, INFORMATION PROCESSING METHOD, AND INFORMATION PROCESSING PROGRAM

(71) Applicant: FUJIFILM CORPORATION, Tokyo (JP)

(72) Inventors: Takeshi FUKUDA, Kanagawa (JP); Haruyasu NAKATSUGAWA, Kanagawa (JP)

(21) Appl. No.: 16/950,924

(22) Filed: Nov. 18, 2020

(30) Foreign Application Priority Data
Nov. 20, 2019 (JP) 2019-210021

(51) Int. Cl. G16H 50/20; G16H 10/20; G16H 10/60; G06Q 10/02

(52) U.S. Cl. CPC G16H 50/20; G06Q 10/02; G16H 10/60; G16H 10/20

(57) ABSTRACT

An information processing device includes a CPU and a storage unit that stores a command executable by the CPU. The CPU acquires breed information representing a breed of a pet and symptom information on a symptom of the pet, predicts a disease or external injury suffered by the pet based on the acquired breed information and symptom information, and extracts an animal hospital that is able to handle the disease or external injury of the pet based on a prediction result.

### CROSS-REFERENCE TO RELATED APPLICATIONS

The present application claims priority under 35 U.S.C. § 119 to Japanese Patent Application No. 2019-210021, filed on Nov. 20, 2019. The above application is hereby expressly incorporated by reference, in its entirety, into the present application.

### BACKGROUND

### 1. Technical Field

The present disclosure relates to an information processing device, an information processing system, an information processing method, and an information processing program.

### 2. Description of the Related Art

Even though an animal suffers from a disease or receives an external injury, it is often difficult for the animal itself to inform an owner of a symptom or the like. Therefore, there is known a technique of introducing an animal hospital for medical treatment of the animal according to a type and an age of the animal. For example, JP2002-342461A discloses a technique in which an operator of an emergency center selects an animal hospital and a transport vehicle for transporting an animal to the animal hospital by referring to information on the animal such as the type and the age of the animal.

### SUMMARY

By the way, in a case where it is difficult for the animal itself to inform the owner of the symptom or the like as described above, the owner is difficult to determine a state of the symptom of the animal. In the case of the animal hospital, it may be more difficult to determine whether or not it is possible to handle a disease suffered by the animal or a received external injury, as compared with a hospital for a person. Therefore, the owner may be uncertain about whether to take the animal to the animal hospital or hesitate to take the animal to the animal hospital.

In a case where the owner is late to take the animal to the animal hospital, a start of the medical treatment is delayed. In the case of the animal, aging progresses faster than the person particularly, and thus the disease may progress more rapidly. Therefore, it is desired to start appropriate medical treatment early.

However, the technique described in JP2002-342461A is a technique intended for an animal with high acute urgency and is not a sufficient technique for starting the medical treatment early.

The present disclosure has been made in view of the above circumstances and provides an information processing device, an information processing system, an information processing method, and an information processing program that allow an animal to receive appropriate medical treatment early.

To achieve the above object, an information processing device according to a first aspect of the present disclosure comprises at least one processor and a memory that stores a command executable by the processor. The processor acquires breed information representing a breed of a subject, which is an animal, and symptom information on a symptom of the subject, predicts a disease or external injury suffered by the subject based on the acquired breed information and symptom information, and extracts an animal hospital that is able to handle the disease or external injury of the subject based on a prediction result.

In an information processing device according to a second aspect of the present disclosure, the processor asks a question about the symptom or external injury to an owner of the subject, receives an answer to the question, and acquires the symptom information based on the received answer, in the information processing device according to the first aspect.

In an information processing device according to a third aspect of the present disclosure, the processor further presents the prediction result, in the information processing device according to the first aspect.

In an information processing device according to a fourth aspect of the present disclosure, the prediction of the disease or external injury suffered by the subject includes prediction of a state of the disease or a state of the external injury, in the information processing device according to the first aspect.

In an information processing device according to a fifth aspect of the present disclosure, the processor presents the extracted animal hospital as the handling animal hospital, in the information processing device according to the first aspect.

In an information processing device according to a sixth aspect of the present disclosure, the processor acquires information indicating a regular animal hospital for the subject and presents the regular animal hospital for the subject as the handling animal hospital in a case where the handling animal hospital includes the regular animal hospital for the subject, in the information processing device according to the fifth aspect.

In an information processing device according to a seventh aspect of the present disclosure, the processor acquires information indicating a current position or a residence place as a position of an owner of the subject or the subject and presents a nearby animal hospital as the handling animal hospital, in a case where the handling animal hospital includes at least one of an animal hospital within a predetermined distance from the current position or an animal hospital within a predetermined distance from the residence place as the nearby animal hospital, in the information processing device according to the fifth aspect.

In an information processing device according to an eighth aspect of the present disclosure, the processor presents an animal hospital that specializes in medical care as the handling animal hospital, in a case where the handling animal hospital includes at least one of an animal hospital that specializes in medical care for the predicted disease or external injury suffered by the subject or an animal hospital that specializes in medical care for a breed represented by the breed information as the animal hospital that specializes in medical care, in the information processing device according to the fifth aspect.

In an information processing device according to a ninth aspect of the present disclosure, the processor extracts an animal hospital having a device used for medical care for the predicted disease or external injury suffered by the subject as the handling animal hospital, in a case where a device of an animal hospital that is used for the medical care is registered in advance, in the information processing device according to the first aspect.

In an information processing device according to a tenth aspect of the present disclosure, the processor receives designation of an animal hospital to receive medical care at a presented animal hospital, makes a reservation for the designated animal hospital for the medical care, and provides the prediction result to the animal hospital of a reservation destination, in the information processing device according to the first aspect.

In order to achieve the above object, an information processing system according to an eleventh aspect of the present disclosure comprises a terminal device owned by an owner of an animal which is a subject and the information processing device according to the present disclosure, which is connected to the terminal device through a network and acquires the breed information and the symptom information from the terminal device.

In order to achieve the above object, a non-transitory computer-readable storage medium storing an information processing method according to a twelfth aspect of the present disclosure executed by a computer comprises acquiring breed information representing a breed of a subject, which is an animal, and symptom information on a symptom of the subject, predicting a disease or external injury suffered by the subject based on the acquired breed information and symptom information, extracting an animal hospital that is able to handle the disease or external injury of the subject based on a prediction result, and presenting the extracted animal hospital.

In order to achieve the above object, an information processing program according to a thirteenth aspect of the present disclosure causing a computer to execute the following processing of acquiring breed information representing a breed of a subject, which is an animal, and symptom information on a symptom of the subject, predicting a disease or external injury suffered by the subject based on the acquired breed information and symptom information, and extracting an animal hospital that is able to handle the disease or external injury of the subject based on a prediction result.

According to the present disclosure, an animal is allowed to receive appropriate medical treatment early.

### BRIEF DESCRIPTION OF THE DRAWINGS

Exemplary embodiments according to the technique of the present disclosure will be described in detail based on the following figures, wherein:

FIG. 1 is a block diagram showing an example of a configuration of an information processing system.

FIG. 2 is a block diagram showing an example of a hardware configuration of an information processing device.

FIG. 3 is a table for describing an example of animal hospital information.

FIG. 4 is a block diagram showing an example of a functional configuration of the information processing device.

FIG. 5 is a table showing an example of a prediction result based on a breed and a symptom.

FIG. 6 is a flowchart showing an example of animal hospital introduction processing executed by the information processing device.

FIG. 7 is a diagram showing an example of an information input form.

FIG. 8 is a diagram showing an example of a symptom information input form of basic symptom information.

FIG. 9 is a diagram showing an example of the symptom information input form for acquiring a symptom appearing in excrement.

FIG. 10 is a diagram showing an example of the symptom information input form for acquiring a symptom appearing on an appearance of a pet.

FIG. 11 is a diagram showing an example of the symptom information input form for acquiring a symptom appearing on a cry of a pet.

FIG. 12A is a diagram showing an example of displayed introduction information and prediction result information.

FIG. 12B is a diagram showing another example of displayed introduction information and prediction result information.

### DETAILED DESCRIPTION

Hereinafter, an embodiment for implementing a technique of the present disclosure will be described in detail with reference to drawings. An “animal” in this specification means animals, such as a dog and a cat, excluding “person”. In the following embodiment, an embodiment in which a pet is employed as an example of a subject animal will be described. In the following embodiment, a “breed” of the animal includes a concept of species such as “dog” and “cat” in addition to the breed such as “dog breed”.

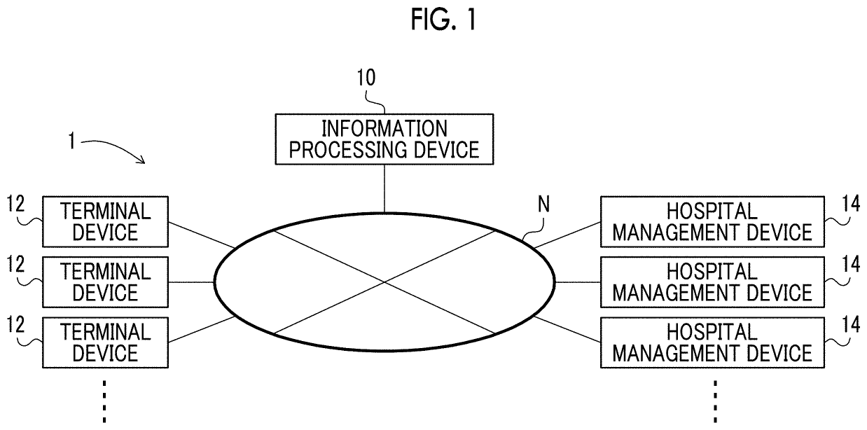

First, an information processing system 1 according to the present embodiment will be described with reference to FIG. 1. FIG. 1 is a block diagram representing an example of a configuration of the information processing system 1 according to the present embodiment. As shown in FIG. 1, the information processing system 1 according to the present embodiment comprises an information processing device 10, a plurality (three in FIG. 1 as an example) of terminal devices 12, and a plurality (three in FIG. 1 as an example) of hospital management devices 14. The information processing device 10 and the plurality of terminal devices 12 each are connected to a network N and can communicate with each other through the network N. The information processing device 10 and the plurality of hospital management device 14 each are connected to the network N and can communicate with each other through the network N.

The information processing device 10 is, for example, a cloud server constructed on the cloud. The information processing device 10 may be a server computer or the like installed in an animal hospital or the like. The terminal device 12 is, for example, a terminal device owned by an owner of the pet which is the subject. Examples of the terminal device 12 include a smartphone and a tablet computer. The hospital management device 14 is installed in, for example, the animal hospital and is used to manage an electronic medical record, manage a reservation for medical care, manage various devices, and the like in the installed animal hospital. Examples of the hospital management device 14 include a personal computer and a tablet computer.

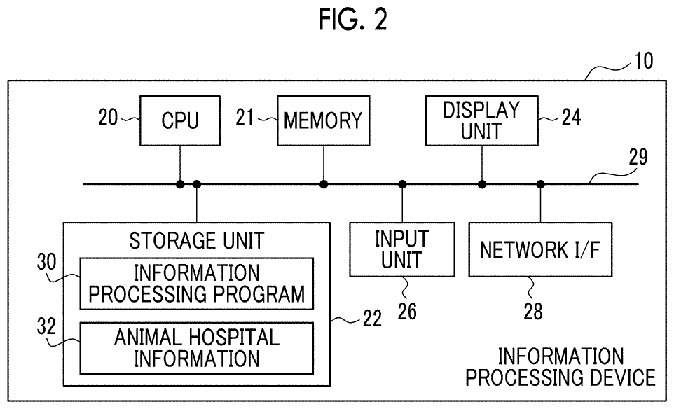

Next, an example of a hardware configuration of the information processing device 10 according to the present embodiment will be described with reference to FIG. 2. As shown in FIG. 2, the information processing device 10 includes a central processing unit (CPU) 20, a memory 21 as a temporary storage region, and a storage unit 22 which is a nonvolatile memory. The CPU 20 according to the present embodiment is an example of a processor according to the present disclosure, and the storage unit 22 is an example of a memory according to the present disclosure.

The information processing device 10 includes a display unit 24 such as a liquid crystal display, an input unit 26 such as a keyboard or a mouse, and a network interface (I/F) 28 connected to the network N. The display unit 24 and the input unit 26 may be integrated as a touch panel display. The CPU 20, the memory 21, the storage unit 22, the display unit 24, the input unit 26, and the network I/F 28 are connected to a bus 29 communicably with each other.

The storage unit 22 is formed by a hard disk drive (HDD), a solid state drive (SSD), a flash memory, and the like. The storage unit 22 as a storage medium stores an information processing program 30. The CPU 20 reads the information processing program 30 from the storage unit 22, develops the program in the memory 21, and executes the developed information processing program 30.

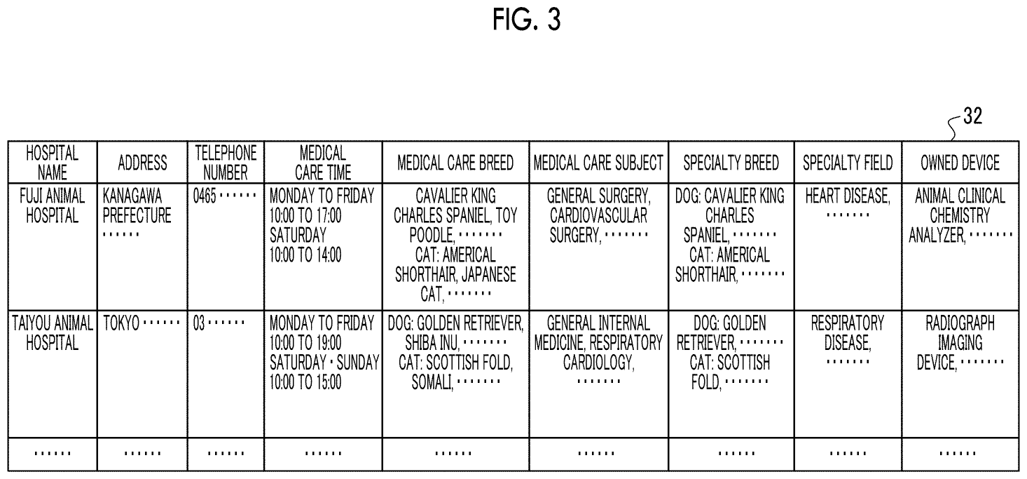

Animal hospital information 32 is stored in the storage unit 22 according to the present embodiment. The animal hospital information 32 according to the present embodiment is information on the animal hospital in which the hospital management device 14 is installed. FIG. 3 shows an example of the animal hospital information 32 of the present embodiment. As shown in FIG. 3, as an example, the animal hospital information 32 according to the present embodiment includes information of “hospital name”, “address”, “telephone number”, “medical care time”, “medical care breed”, “medical care subject”, “specialty breed”, “specialty field”, and “owned device” for each animal hospital in which the hospital management device 14 is installed. The information included in the animal hospital information 32 is not limited to the above. For example, the animal hospital information 32 may include information representing whether a house call is possible, or the like.

The “hospital name” among the animal hospital information 32 is information representing the name of the animal hospital. The “address” is information representing an address (location) of the animal hospital. The “telephone number” is information representing a telephone number of the animal hospital. The “medical care time” is information representing a medical care time of the animal hospital.

The “medical care breed” is information representing the breed of pet for which the medical care can be performed at the animal hospital. The information representing the “medical care breed” in the animal hospital information 32 illustrated in FIG. 3 includes the dog breed. However, the information is not limited to this embodiment and may be only a race, for example. The “medical care subject” is information representing a subject for which the medical care can be performed at the animal hospital.

The “specialty breed” is information representing the breed that the animal hospital specializes in. Specifically, the “specialty breed” is information representing a breed specialized in the animal hospital, a breed with a large number of affected animals, and the like. The “specialty breed” may be only the race, similarly to the “medical care breed” described above. However, it is preferable to include a detailed classification such as the dog breed. The “specialty field” is information representing a field of disease or external injury in which the animal hospital specializes in the medical care. Specifically, the “specialty field” is information representing a field specialized in the animal hospital, a field often dealt with as a case, and the like.

The “owned device” is information representing a device owned by the animal hospital and used for the medical care. In the present embodiment, the “medical care” includes an examination. Therefore, the “owned device” includes a device used for the examination of the pet, a device used for medical treatment, and the like.

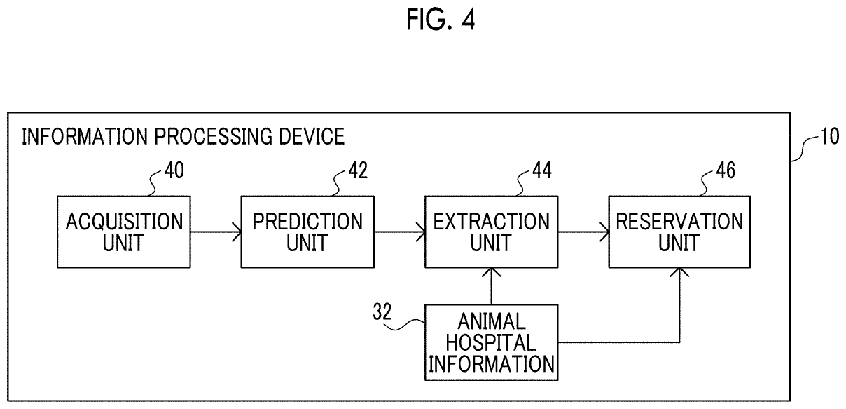

Next, a functional configuration of the information processing device 10 according to the present embodiment will be described with reference to FIG. 4. As shown in FIG. 4, the information processing device 10 includes an acquisition unit 40, a prediction unit 42, an extraction unit 44, and a reservation unit 46. The CPU 20 executes the information processing program 30 to function as the acquisition unit 40, the prediction unit 42, the extraction unit 44, and the reservation unit 46.

The acquisition unit 40 acquires pet information on the pet. As described below in detail, the suffered disease or external injury may differ depending on the breed even though pets show the same symptom. Therefore, the pet information is not limited as long as the information is information on the pet including at least breed information representing the breed of the pet. The pet information preferably includes information representing the name of the pet, information representing an age, information representing sex, and the like, in addition to the breed information, and more preferably includes information for identifying a regular animal hospital for the pet and information representing an address of the owner (pet).

The acquisition unit 40 acquires symptom information on the symptom of the pet. As an example, the acquisition unit 40 according to the present embodiment employs a chatbot that acquires the symptom information from the hospital management device 14 of the user in an interactive manner. The acquisition unit 40 detects a specific word, phrase, or the like from information input from the owner and outputs a question prepared according to the specific word, phrase, or the like to the terminal device 12 owned by the pet owner. The acquisition unit 40 may be a chatbot that uses artificial intelligence (AI). For example, the acquisition unit 40 may employ the chatbot by AI using a learned model generated to output the question related to the symptom, based on a combination of a word or a phrase related to the symptom and the breed by performing learning with the combination of the word or phrase related to the symptom, a genetic disease specific to the breed, a frequent disease specific to the breed, and the like and the question, as learning data (also referred to as teacher data). For example, an error back propagation method may be employed as an algorithm of the learning in this case.

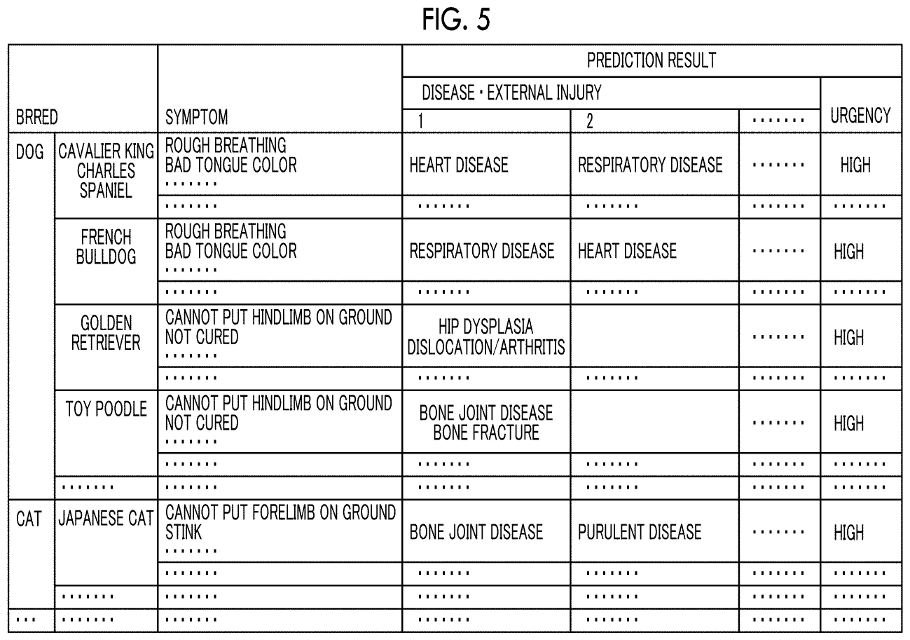

The prediction unit 42 predicts the disease or external injury suffered by the pet based on the breed information included in the pet information and the symptom information acquired by the acquisition unit 40. As described above, the suffered disease or external injury may differ depending on the breed even though the pets show the same symptom. Therefore, prediction unit 42 predicts the disease or external injury suffered by the pet based on the breed represented by the breed information of the pet and the symptom represented by the symptom information. FIG. 5 shows an example of a prediction result based on the breed and the symptom. As shown in FIG. 5, the prediction performed by the prediction unit 42 according to the present embodiment includes prediction of the disease or the external injury and prediction of urgency. The prediction of the disease or the external injury includes prediction of a state of the disease or the external injury. The prediction unit 42 predicts a plurality of diseases or external injuries in descending order of suffering possibility.

The prediction of the disease or external injury of the pet in the prediction unit 42 according to the present embodiment will be described in detail with reference to FIG. 5. As shown in FIG. 5, prediction results differ between a case where the breed is “dog” and “Cavalier King Charles Spaniel” and a case where the breed is “dog” and “French Bulldog”, even though the dogs show the same symptoms of “rough breathing” and “bad tongue color”. The “Cavalier King Charles Spaniel” is known as a breed having a heart disease frequently, and the symptoms of “rough breathing” and “bad tongue color” are symptoms in aggravating stage and urgency of the heart disease. Therefore, the prediction unit 42 predicts “heart disease” as a disease most likely to be suffered (first prediction result) and predicts that the urgency is high. The above symptom also occurs in a respiratory disease and the like. Therefore, the prediction unit 42 predicts that the possibility of “respiratory disease” is also high (second prediction result). On the other hand, the “French Bulldog” is known as a breed having brachycephalic airway syndrome frequently, and the symptoms of “rough breathing” and “bad tongue color” are symptoms in incomplete airway obstruction and urgency. Therefore, the prediction unit 42 predicts “respiratory disease” as the disease most likely to be suffered (first prediction result) and predicts that the urgency is high. The above symptom also occurs in the heart disease and the like. Therefore, the prediction unit 42 predicts that the possibility of “heart disease” is also high (second prediction result).

As shown in FIG. 5, prediction results differ between a case where the breed is “dog” and “Golden Retriever” and a case where the breed is “dog” and “Toy Poodle”, even though the dogs show the same symptoms of “cannot put hindlimb on the ground” and “not cured”. The “Golden Retriever” is known as a breed having hip dysplasia frequently, and the symptoms of “cannot put hindlimb on the ground” and “not cured” are symptoms in abnormality of a bone. Therefore, the prediction unit 42 predicts “dislocation/arthritis due to hip dysplasia” as the disease most likely to be suffered (first prediction result) and predicts that the urgency is high. On the other hand, “Toy Poodle” is known as a breed having a bone fracture frequently, and the symptoms of “cannot put hindlimb on the ground” and “not cured” are symptoms in abnormality of a bone. Therefore, the prediction unit 42 predicts the “bone fracture of bone joint disease” as the disease most likely to be suffered (first prediction result) and predicts that the urgency is high.

A specific prediction method in the prediction unit 42 is not particularly limited. For example, a prediction method may be employed in which the prediction is performed based on information representing a correspondence relationship between the breed and symptom and the prediction result as shown in FIG. 5. For example, a prediction method may be employed in which the prediction is performed based on information representing a correspondence relationship between the breed, the frequent disease according to the breed, and the symptom.

The prediction unit 42 may perform the prediction using AI. For example, the prediction unit 42 may employ AI using a learned model generated to output the prediction result of the suffered disease or external injury, based on a combination of the breed and the symptom by performing learning with a combination of the breed, the symptom, and the suffered disease or external injury, as learning data (also referred to as teacher data). For example, an error back propagation method may be employed as an algorithm of the learning in this case.

The extraction unit 44 extracts an animal hospital that is able to handle the disease or external injury of the pet based on the prediction result of the prediction unit 42. Specifically, the extraction unit 44 according to the present embodiment refers to the animal hospital information 32 to extract the animal hospital in which all diseases or external injuries included in the prediction result are included in the medical care subject and the breed of the pet is included in the medical care breed. A device used for the medical care owned by each animal hospital is registered in the animal hospital information 32 in advance. Therefore, the extraction unit 44 extracts the animal hospital having a device used for the medical care of the disease or external injury included in the prediction result.

The extraction unit 44 presents the extracted animal hospital to the terminal device 12 to introduce the extracted animal hospital to the owner of the pet. Specifically, the extraction unit 44 sets priority for extracted animal hospitals and presents to the terminal device 12 in order from the animal hospital with the highest priority to output introduction information for introducing the animal hospital to the owner of the pet to the terminal device 12 owned by the owner. As an example, in a case where there is a regular animal hospital for the pet, the extraction unit 44 presents the priority of the regular animal hospital, as the highest (first). As described above, the priority of the regular animal hospital is raised, and thus it is possible to preferentially introduce the animal hospital that the owner can easily visit.

Further, the extraction unit 44 presents the priority of the animal hospital near a current position or a residence place of the pet or the owner as a next order (second). As described above, the priority of the nearby animal hospital is raised, and thus it is possible to preferentially introduce the animal hospital that the owner can easily visit.

The extraction unit 44 presents the priority of at least one of the animal hospital that specializes in the medical care for all diseases or external injuries included in the prediction result or the animal hospital that specializes in the medical care for the breed of the pet, as a next order (third). In a case where the animal hospital that specializes in the medical care for all diseases or external injuries included in the prediction result is not included in the extracted animal hospitals, the priority of the animal hospital that specializes in the medical care for the disease or external injury of at least the first prediction result may be the next order (third). As described above, the priority of the animal hospital that specializes in the medical care is raised, and thus it is possible to preferentially introduce the animal hospital which is more suitable for the medical care.

The priority of presenting the animal hospital is not limited to the present embodiment, and the owner may set the priority, for example.

The reservation unit 46 receives, from the terminal device 12, designation for receiving the medical care at the animal hospital presented by the extraction unit 44 and makes a reservation for the designated animal hospital for the medical care. In a case where the reservation is made, the reservation unit 46 provides the prediction result of the prediction unit 42 to the animal hospital of the reservation destination.

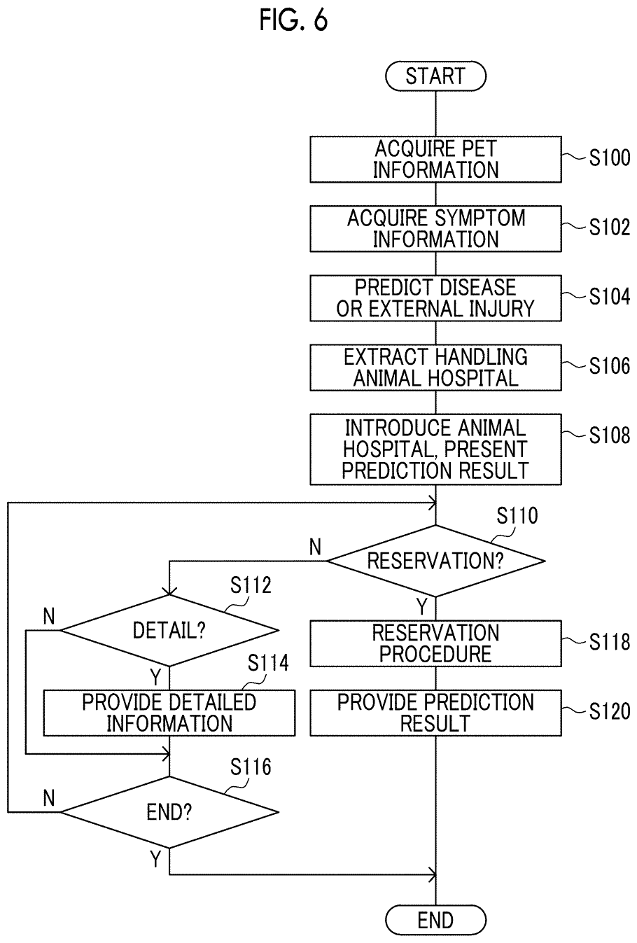

Next, an action of the information processing device 10 according to the present embodiment will be described with reference to FIG. 6. The CPU 20 executes the information processing program 30 to execute animal hospital introduction processing shown in FIG. 6. The animal hospital introduction processing shown in FIG. 6 is executed, for example, in a case where execution designation is received from the terminal device 12.

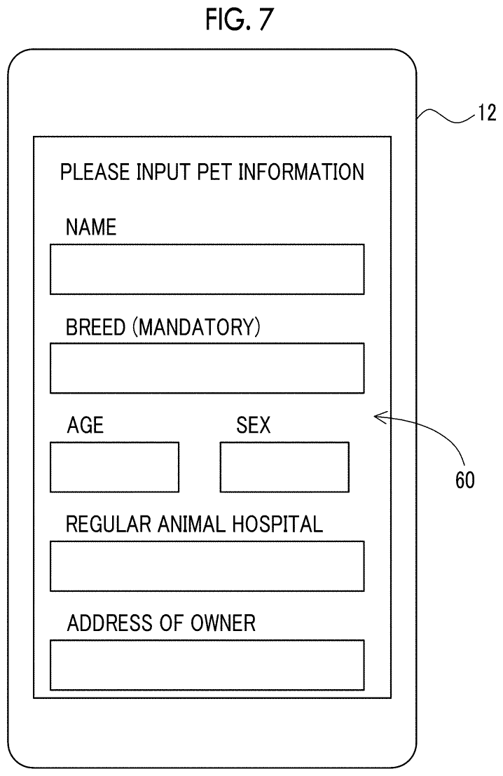

In step S100 in FIG. 6, the acquisition unit 40 acquires the pet information from the terminal device 12 owned by the owner, as described above. Specifically, the acquisition unit 40 outputs a pet information input form for the owner to input the pet information to the terminal device 12. FIG. 7 shows an example of a pet information input form 60 displayed on the terminal device 12. The pet information input form 60 is an example of the pet information input form for inputting the pet breed as a mandatory input item, and pet name, age, sex, regular animal hospital, and address of the owner as other input items. The owner inputs each item as the pet information according to the pet information input form 60 displayed on the terminal device 12. The input pet information is output from the terminal device 12. The acquisition unit 40 acquires the pet information output from the terminal device 12.

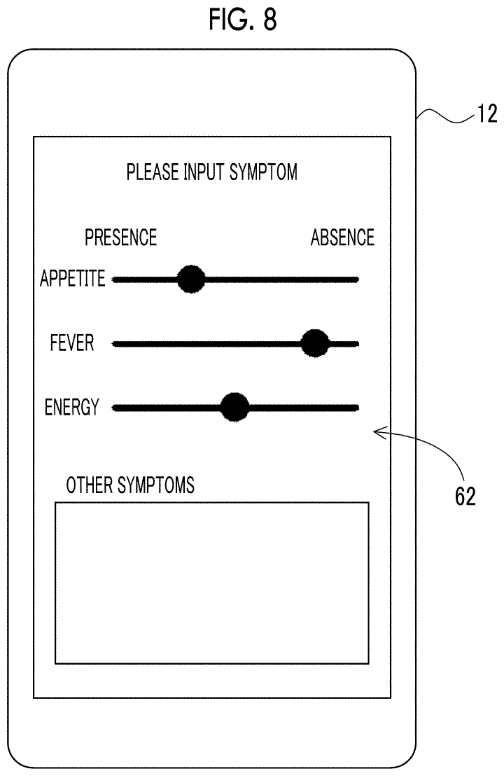

In next step S102, the acquisition unit 40 acquires the symptom information from the terminal device 12, as described above. Specifically, the acquisition unit 40 outputs a symptom information input form for the owner to input symptom information to the terminal device 12. As described above, the acquisition unit 40 according to the present embodiment acquires the symptom information in a question format. Therefore, the acquisition unit 40 first outputs the symptom information input form of basic symptom information to the terminal device 12. FIG. 8 shows an example of the symptom information input form 62 of the basic symptom information displayed on the terminal device 12. The symptom information input form 62 is an example of a symptom information input form for inputting presence or absence of each of appetite, fever, and energy in the pet and other symptoms. The owner inputs each item as the symptom information according to the symptom information input form 62 displayed on the terminal device 12. The symptom is input in a free word format as for the other symptoms in the symptom information input form 62. The input pet information is output from the terminal device 12. The acquisition unit 40 acquires the symptom information output from the terminal device 12. The acquired symptom information is temporarily stored in the information processing device 10.

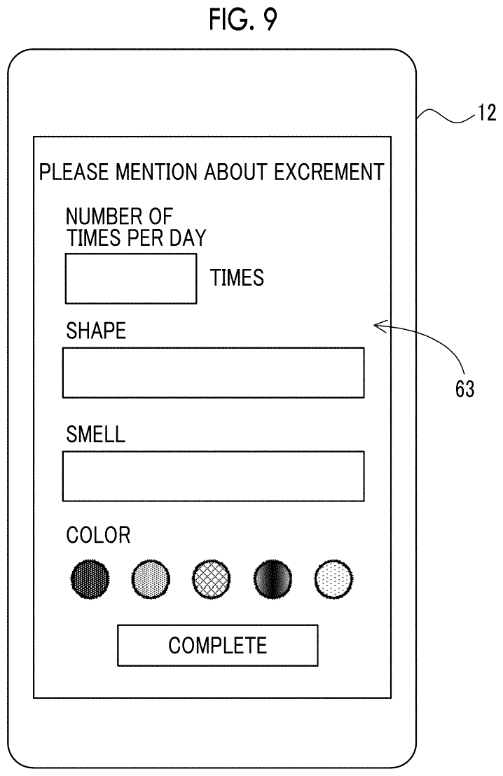

In a case where the basic symptom information is acquired by the symptom information input form 62, the prediction unit 42 analyzes the item (word, sentence, or the like) acquired as the other symptoms and outputs the symptom information input form prepared according to a specific word, phrase, or the like to the terminal device 12. For example, in a case where the symptom information acquired by the symptom information input form 62 includes a word, a phrase, or the like related to excrement such as “feces” and “urine”, the symptom information input form for acquiring the symptom appearing in the excrement is output to the terminal device 12 as shown in FIG. 9. FIG. 9 shows an example of the symptom information input form 63 for acquiring the symptom appearing in the excrement. The symptom information input form 63 is an example of a symptom information input form for inputting each of the number, shape, odor, and color of the excrement of the pet. The owner inputs each item as the symptom information according to the symptom information input form 63 displayed on the terminal device 12. The input pet information is output from the terminal device 12. The acquisition unit 40 acquires the symptom information output from the terminal device 12. The acquired symptom information is temporarily stored in the information processing device 10.

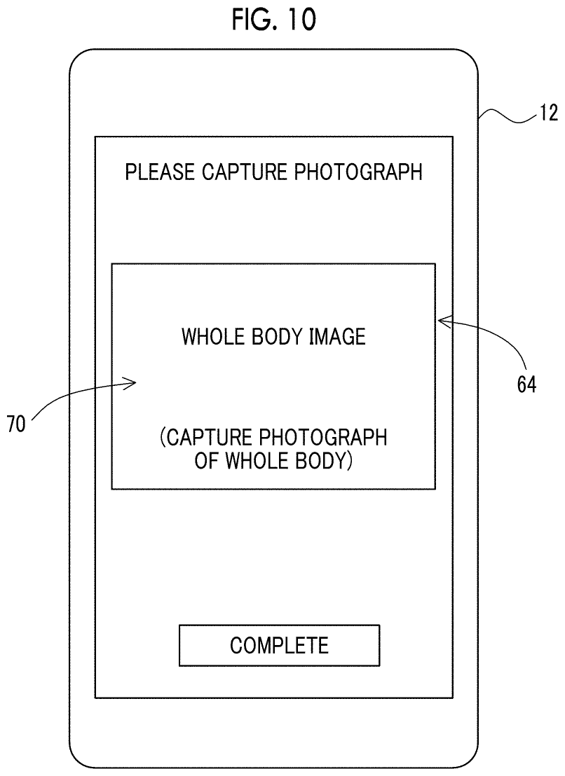

For example, in a case where the symptom information acquired by the symptom information input form 62 includes a word, a phrase, or the like related to the appearance of the pet such as “injury”, “burn”, and “skin”, the symptom information input form for acquiring the symptom appearing on the appearance of the pet is output to the terminal device 12 as shown in FIG. 10. FIG. 10 shows an example of a symptom information input form 64 for acquiring the symptom appearing on the appearance of the pet. The symptom information input form 64 is an example of a symptom information input form for inputting a whole body image (photograph of whole body) as an image (photograph) of the appearance of the pet.

The owner inputs the image of the pet as the symptom information according to the symptom information input form 64 displayed on the terminal device 12. As a specific example, in the terminal device 12 according to the present embodiment, in a case where the image of the pet is input according to the symptom information input form 64, the owner designates (clicks) an image input region 70 of the symptom information input form 64 displayed on the terminal device 12. In a case where the terminal device 12 detects that the image input region 70 is designated, the terminal device 12 activates an imaging unit (not shown) included in the terminal device 12 to perform a mode switching to an imaging mode. With the mode switching to the imaging mode, the display switches from the symptom information input form 64 to a camera screen, and the owner captures the image of the pet with the imaging unit. In a case where the image is captured by the imaging unit and the captured image (photograph) is acquired, the display is switched from the camera screen to the symptom information input form 64, and the captured image of the pet is displayed in the image input region 70. The input pet information is output from the terminal device 12. The acquisition unit 40 acquires the symptom information output from the terminal device 12. The acquired symptom information is temporarily stored in the information processing device 10. In a case where the captured image (photograph) is acquired as the symptom information as described above, the symptom information input form 64 may display, as an initial state, an example of the captured image as a reference image in the image input region 70, as a reference of an input method of the symptom information.

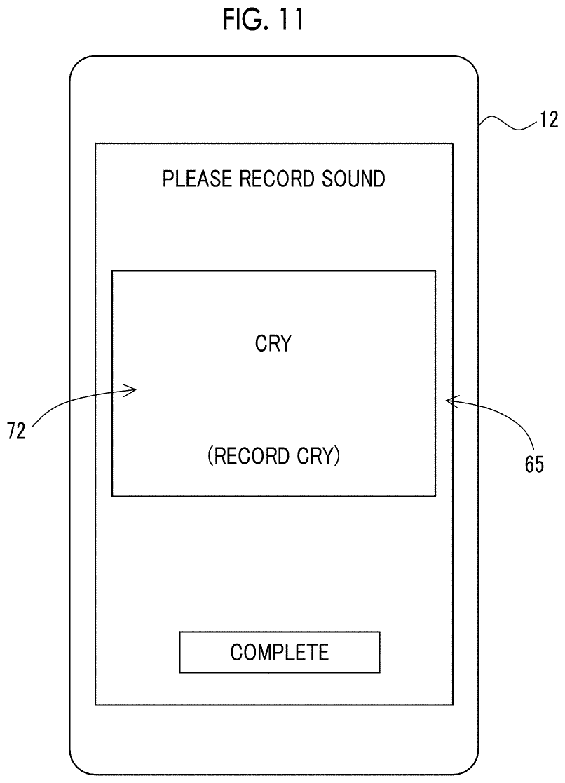

For example, in a case where the symptom information acquired by the symptom information input form 62 includes a word, a phrase, or the like related to a cry of the pet such as “crying” and “sound”, the symptom information input form for acquiring the symptom appearing on the cry of the pet is output to the terminal device 12 as shown in FIG. 11. FIG. 11 shows an example of a symptom information input form 65 for acquiring the symptom appearing on the cry of the pet. The symptom information input form 65 is an example of a symptom information input form for inputting the cry of the pet.

The owner inputs the cry of the pet as the symptom information according to the symptom information input form 65 displayed on the terminal device 12. As a specific example, in the terminal device 12 according to the present embodiment, in a case where the cry of the pet is input according to the symptom information input form 65, the owner designates (clicks) a cry input region 72 of the symptom information input form 65 displayed on the terminal device 12. In a case where the terminal device 12 detects that the cry input region 72 is designated, the terminal device 12 performs a mode switching to a sound collection mode. In a case where the mode is switched to the sound collection mode, the terminal device 12, for example, records the sound collected by a sound collection device (not shown) provided in the terminal device 12 for a predetermined time such as one minute. In a case where the recording ends, the sound collection mode ends. The input pet information is output from the terminal device 12. The acquisition unit 40 acquires the symptom information output from the terminal device 12. The acquired symptom information is temporarily stored in the information processing device 10.

As described above, the acquisition unit 40 repeats the question, specifically, the output of the symptom information input form and acquisition of the symptom information input by the owner to acquire the symptom information.

In next step S104, the prediction unit 42 predicts the disease or external injury suffered by the pet based on the breed information included in the pet information acquired in step S100 and the symptom information acquired in step S102, as described above. The processing in steps S102 and S104 may be repeated. In other words, the acquisition of the symptom information by the acquisition unit 40 and the prediction of the disease or external injury suffered by the pet by the prediction unit 42 may be repeated a plurality of times to confirm the prediction.

In next step S106, the extraction unit 44 extracts the animal hospital that is able to handle the disease or external injury of the pet based on the prediction result in step S104, as described above.

In next step S108, the extraction unit 44 introduces the extracted animal hospital to the terminal device 12 to introduce the animal hospital to the owner, as described above. Specifically, the priority is specified for the extracted animal hospitals as described above, and the animal hospitals are presented in descending order of the specified priority. More specifically, in a case where the pet information acquired in step S100 includes the regular animal hospital and the regular animal hospital is included in the extracted animal hospitals, the extraction unit 44 sets the priority of the regular animal hospital as the highest (first). In a case where the pet information acquired in step S100 includes the address of the owner, the extraction unit 44 refers to the “address” of the animal hospital information 32 to set the priority of a nearby animal hospital within a predetermined distance from the acquired address as the next order (second). Instead of the address of the owner, the priority of the animal hospital near the current position of the owner or pet may be set as the next order (second). In this case, position information representing the current position of the terminal device 12 may be acquired from the terminal device 12 regarding the current position of the owner or pet as the current position of the terminal device 12. The extraction unit 44 sets the priority of at least one of the animal hospital that specializes in the medical care for all the diseases or external injuries included in the prediction result, which is specified by referring the “specialty field” of the animal hospital information 32, or the animal hospital that specializes in the medical care for the breed of the pet, which is specified by referring the “specialty breed” of the animal hospital information 32, as the next order (third).

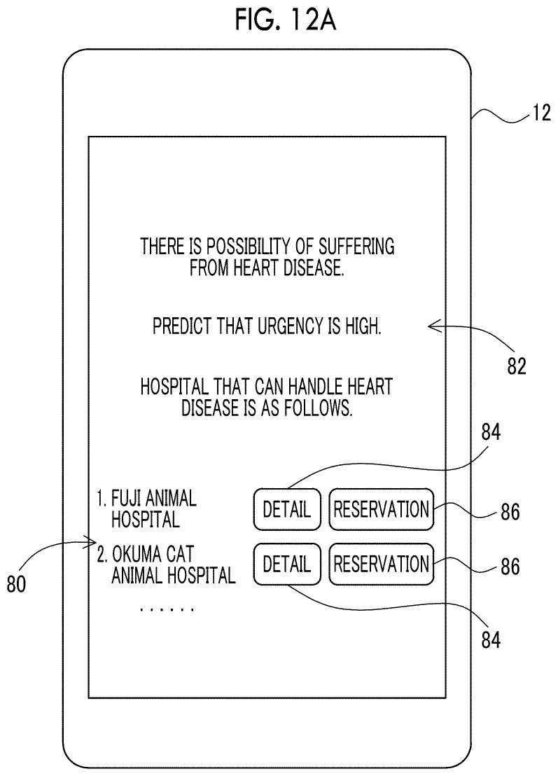

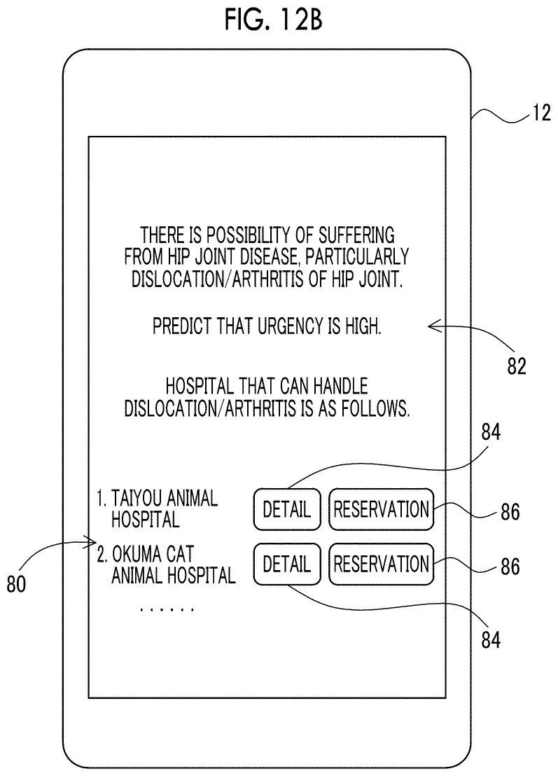

In step S108, the extraction unit 44 presents the prediction result to the owner. Therefore, the extraction unit 44 outputs the introduction information representing the animal hospital to be introduced and prediction result information representing the prediction result to the terminal device 12. FIGS. 12A and 12B show examples of introduction information 80 and prediction result information 82 displayed on the terminal device 12. FIG. 12A shows the examples of the prediction result information 82 representing the prediction result that the pet suffers from the heart disease and the urgency is high and the introduction information 80. FIG. 12B shows the examples of the prediction result information 82 representing the prediction result that the pet suffers from a bone joint disease, particularly dislocation/arthritis of the hip joint and the urgency is high, and the introduction information 80.

As shown in FIGS. 12A and 12B, the terminal device 12 displays a detail button 84 and a reservation button 86 for each hospital to be introduced, in addition to the introduction information 80. In a case where the owner designates the detail button 84, designation information for designating provision of detailed information is output from the terminal device 12 to the information processing device 10. The reservation unit 46 acquires the detailed information on the animal hospital, such as an address of the animal hospital or a medical care time corresponding to the detail button 84 designated by the owner, from the animal hospital information 32 and outputs the information to the terminal device 12. The terminal device 12 displays the detailed information on the animal hospital, which is input from the information processing device 10.

In a case where the owner designates the reservation button 86, designation information for designating the reservation is output from the terminal device 12 to the information processing device 10. The reservation unit 46 makes a reservation for the animal hospital corresponding to the reservation button 86 designated by the owner.

In next step S110, the reservation unit 46 determines whether or not the designation information for designating the reservation is input from the information processing device 10. In a case where the designation information for designating the reservation is not input, the determination in step S110 is negative and the processing proceeds to step S112.

In step S112, the reservation unit 46 determines whether or not the designation information for designating the provision of the detailed information is input from the terminal device 12. In a case where the designation information for designating the provision of the detailed information is not input, the determination in step S112 is negative and the processing proceeds to step S116. On the other hand, in a case where the designation information for designating the provision of the detailed information is input, the determination in step S112 is affirmative and the processing proceeds to step S114.

In step S114, the extraction unit 44 outputs the detailed information on the animal hospital corresponding to the detail button 84 designated by the owner to the terminal device 12 to provide the information to the owner, as described above.

In next step S116, the reservation unit 46 determines whether or not to end the animal hospital introduction processing. For example, in a case where designation to end the animal hospital introduction processing is received from the terminal device 12 and in a case where a predetermined end condition, such as a case where the terminal device 12 is powered off, is satisfied, the determination in step S116 is affirmative and the animal hospital introduction processing ends. On the other hand, the determination in step S116 is negative and the processing returns to step S110 until the predetermined end condition is satisfied.

In a case where the designation information for designating the reservation is input in step S110, the determination is affirmative and the processing proceeds to step S118. In step S118, the reservation unit 46 accesses the hospital management device 14 of the animal hospital corresponding to the reservation button 86 designated by the owner to perform a procedure for reservation of the medical care for the pet.

In next step S120, the reservation unit 46 outputs the prediction result in step S104 to the hospital management device 14 of the reserved animal hospital to provide the prediction result. In a case where the processing in step S120 ends, the animal hospital introduction processing ends.

As described above, the information processing device 10 according to the present embodiment comprises the CPU 20 and the storage unit 22 that stores a command executable by the CPU 20. The CPU 20 acquires the breed information representing the breed of the pet and the symptom information on the symptom of the pet, predicts the disease or external injury suffered by the pet based on the acquired breed information and symptom information, and outputs the animal hospital that is able to handle the disease or external injury of the pet based on the prediction result.

As described above, in the information processing device 10 according to the present embodiment, it is possible to extract the animal hospital that is able to handle the disease or external injury suffered by the pet. As a result, the extracted animal hospital can be presented to the owner. The pet is allowed to receive appropriate medical treatment early. Since early receiving of the appropriate medical treatment is possible, it is possible to suppress the delay in the start of the medical treatment even for the animal that aging progresses faster than a person and the disease progresses relatively rapidly.

It is sufficient for the owner to input the pet information including the breed information and the symptom information into each input form (pet information input form and symptom information input form). Therefore, it is possible to easily obtain the information on the animal hospital to which the pet is taken and find out the disease or external injury suffered by the pet.

In the present embodiment, the prediction unit 42 predicts the disease or external injury suffered by the pet based on the breed information and the symptom information, but the present disclosure is not limited to this embodiment. The prediction unit 42 may make the prediction by further using another piece of information that influences the disease or external injury of the pet. The prediction unit 42 may make the prediction based on, for example, pet information such as age information representing the age of the pet, sex information representing sex, and castration information representing presence or absence of castration (contraception), in addition to the breed information and the symptom information.

In the present embodiment, the pet information and the symptom information are acquired by inputting the character or the like by the owner, but the present disclosure is not limited to this embodiment. For example, at least one of the acquisition of the pet information or the acquisition of the symptom information may be performed by further using sound input, or the acquisition unit 40 of the information processing device 10 and the owner may interact with each other to acquire various types of information.

The following various processors may be used as a hardware structure of a processing unit that executes various types of processing such as each functional unit of the information processing device 10 in the present embodiment. The various processors include a programmable logic device (PLD) which is a processor whose circuit configuration is changeable after manufacturing such as a field-programmable gate array (FPGA), a dedicated electric circuit which is a processor having a circuit configuration exclusively designed to execute specific processing such as an application specific integrated circuit (ASIC), and the like, in addition to the CPU which is a general-purpose processor that executes software (program) to function as various processing units as described above.

One processing unit may be configured by one of the various processors or a combination of two or more processors of the same type or different types (for example, a combination of a plurality of FPGAs or a combination of a CPU and an FPGA). A plurality of processing units may be configured by one processor.

As an example of configuring the plurality of processing units with one processor, first, there is a form in which one processor is configured by a combination of one or more CPUs and software and the processor functions as the plurality of processing units, as represented by computers such as a client and a server. Second, there is a form in which a processor that realizes the functions of the entire system including the plurality of processing units with one integrated circuit (IC) chip is used, as represented by a system-on-chip (SoC) or the like. As described above, the various processing units are configured using one or more of the various processors as the hardware structure.

Further, more specifically, a circuitry combining circuit elements such as semiconductor elements can be used as the hardware structure of the various processors.

In the present embodiment, the information processing program 30 is stored (installed) in the storage unit 22 in advance, but the present disclosure is not limited thereto. The information processing program 30 may be provided in a form of being recorded on a recording medium such as a compact disc read only memory (CD-ROM), a digital versatile disc read only memory (DVD-ROM), and a Universal Serial Bus (USB) memory. The information processing program 30 may be downloaded from an external device through a network.

What is claimed is:

1. An information processing device comprising:
at least one processor; and
a memory that stores a command executable by the processor,
wherein the processor
acquires breed information representing a breed of a subject, which is an animal, and symptom information on a symptom of the subject,
predicts a disease or external injury suffered by the subject based on the acquired breed information and symptom information, and
extracts an animal hospital that is able to handle the disease or external injury of the subject based on a prediction result.

2. The information processing device according to claim 1,
wherein the processor
asks a question about the symptom or external injury to an owner of the subject,
receives an answer to the question, and
acquires the symptom information based on the received answer.

3. The information processing device according to claim 1,
wherein the processor
further presents the prediction result.

4. The information processing device according to claim 1,
wherein the prediction of the disease or external injury suffered by the subject includes prediction of a state of the disease or a state of the external injury.

5. The information processing device according to claim 1,
wherein the processor
presents the extracted animal hospital as the handling animal hospital.

6. The information processing device according to claim 5,
wherein the processor
acquires information indicating a regular animal hospital for the subject, and
presents the regular animal hospital for the subject as the handling animal hospital in a case where the handling animal hospital includes the regular animal hospital for the subject.

7. The information processing device according to claim 5,
wherein the processor
acquires information indicating a current position or a residence place as a position of an owner of the subject or the subject, and
presents a nearby animal hospital as the handling animal hospital, in a case where the handling animal hospital includes at least one of an animal hospital within a predetermined distance from the current position or an animal hospital within a predetermined distance from the residence place as the nearby animal hospital.

8. The information processing device according to claim 5,
wherein the processor
presents an animal hospital that specializes in medical care as the handling animal hospital, in a case where the handling animal hospital includes at least one of an animal hospital that specializes in medical care for the predicted disease or external injury suffered by the subject or an animal hospital that specializes in medical care for a breed represented by the breed information as the animal hospital that specializes in medical care.

9. The information processing device according to claim 1,
wherein the processor
extracts an animal hospital having a device used for medical care for the predicted disease or external injury suffered by the subject as the handling animal hospital, in a case where a device of an animal hospital that is used for the medical care is registered in advance.

10. The information processing device according to claim 1,
wherein the processor
receives designation of an animal hospital to receive medical care at a presented animal hospital,
makes a reservation for the designated animal hospital for the medical care, and
provides the prediction result to the animal hospital of a reservation destination.

11. An information processing system comprising:
a terminal device owned by an owner of an animal which is a subject; and
the information processing device according to claim 1, which is connected to the terminal device through a network and acquires the breed information and the symptom information from the terminal device.

12. An information processing method executed by a computer, comprising:
acquiring breed information representing a breed of a subject, which is an animal, and symptom information on a symptom of the subject;
predicting a disease or external injury suffered by the subject based on the acquired breed information and symptom information;
extracting an animal hospital that is able to handle the disease or external injury of the subject based on a prediction result; and
presenting the extracted animal hospital.

13. A non-transitory computer-readable storage medium storing an information processing program causing a computer to execute the following processing of:
acquiring breed information representing a breed of a subject, which is an animal, and symptom information on a symptom of the subject;
predicting a disease or external injury suffered by the subject based on the acquired breed information and symptom information; and
extracting an animal hospital that is able to handle the disease or external injury of the subject based on a prediction result.
